(19) United States
(12) Patent Application Publication
(10) Pub. No.: US 2009/0229609 A1
(43) Pub. Date: Sep. 17, 2009

(54) Portable, disposable cool air inhaler and methods of treatment using same

(76) Inventor: Frank J. Carrier, Shrewsbury, MA (US)

Correspondence Address: Nields, Lemack & Frame, LLC, 176 E. Main Street, Suite #5, Westborough, MA, 01581, US

(21) Appl. No.: 12/075,741

(22) Filed: Mar. 13, 2008

(51) Int. Cl. A61M 16/00

(52) U.S. Cl. 128/204.15

(57) ABSTRACT

An inhaler having a housing containing a low temperature material. Positioned in the housing is a coil or other pathway isolated from the housing and defining an airflow passageway. The coil and the air inside the coil are cooled by the low temperature material when activated. The low temperature material is normally in an inactive state, and requires activation in order to cool. Activation can be initiated immediately prior to use by flexing a flexible or collapsible member in which an activating agent is stored. Also disclosed is a method of activating a low temperature material by causing the low temperature material to contact an activating agent, and inhaling (or forcing manually or automatically) cool air from the device to cause cool air to enter the airways of a patient.

### BACKGROUND OF THE INVENTION

The present invention relates to inhalation apparatus for the relief from symptoms of various respiratory illnesses or distresses, including postoperative distress, and to methods of treating such symptoms.

There are a number of diseases and conditions that may cause upper respiratory tract symptoms that result in considerable discomfort. Exemplary diseases and conditions include the common cold, croup, laryngitis, Laryngotracheobronchitis, bronchitis, strep throat, mononucleosis, whooping cough (pertussis), respiratory tract infections, respiratory syncytial virus (RSV), flu, pneumonia, allergies, asthma, tonsillitis, etc. Exposure to severe environmental conditions, such as excessive heat during a fire or exposure to toxic gases may result in similar symptoms and cause similar discomfort. In addition, such discomfort may be present after surgery, particularly where anesthesia is involved, or may manifest itself as a consequence of sleep apnea.

Various remedies that attempt to reduce or alleviate such discomfort are available, including vapor rubs, cough drops and lozenges, humidifiers, vaporizers, etc. These remedies, however, suffer from various disadvantages. For example, humidifiers, cool mist vaporizers or ultrasonic nebulizers require confining the patient to a relatively small area, as does the use of a hot shower, which confines the patient to a space that is relatively small and that can be very uncomfortable. Bacteria and fungi can grow in the filters and water tanks of portable and console humidifiers, and can be aerosolized and distributed throughout the room. Cough drops and lozenges can have high sugar content and can be high in calories.

It would therefore be desirable to provide a device that is inexpensive and convenient to use, and that reduces or eliminates the foregoing discomforts without suffering from the drawbacks of the prior art.

### SUMMARY OF THE INVENTION

The problems of the prior art have been overcome by the present invention, which provides portable, recyclable or disposable inhaler device and a method of treating various symptoms associated with the airway and/or throat and/or respiratory system of a patient by cooling the airway and/or throat. The device is particularly designed to reduce swelling and inflammation of, and/or relieve symptoms associated with the swelling and inflammation of the larynx and or upper respiratory track, such as swelling and/or inflammation that result from croup, laryngitis's, laryngotracheobronchitis, sleep apnea, anesthesia, and other diseases and conditions including those mentioned above. The device does not require that the user wear a mask.

In certain embodiments, the device is disposable after a single use, and includes a housing containing a low temperature material. Positioned in the housing is a coil or other pathway isolated from the housing and defining an airflow passageway. The coil, and therefore the air inside the coil, is cooled by the low temperature material. When a patient inhales from the device, air in the airflow passageway cooled (and/or humidified) by the low temperature material flows into the patient's airways.

In certain embodiments, the low temperature material is normally in an inactive state, and requires activation in order to function as a low temperature material. Activation is typically initiated immediately prior to use.

In certain embodiments, an activating agent is stored in a flexible member in fluid communication with the low temperature material. Upon flexing of the flexible member (such as by compression, stretching or twisting), the activating agent is released and comes into contact with the low temperature material, causing an endothermic reaction with the low temperature material.

In its method aspects, the present invention includes activating a low temperature material by causing the low temperature material to contact an activating agent, and inhaling (or forcing manually or automatically) cool and/or moist air from the device to cause cool and/or moist air to enter the airways of a patient.

The device can be hand-held, and is generally suitable for a single one-time use, after which it can be recycled or disposed of.

### BRIEF DESCRIPTION OF THE INVENTION

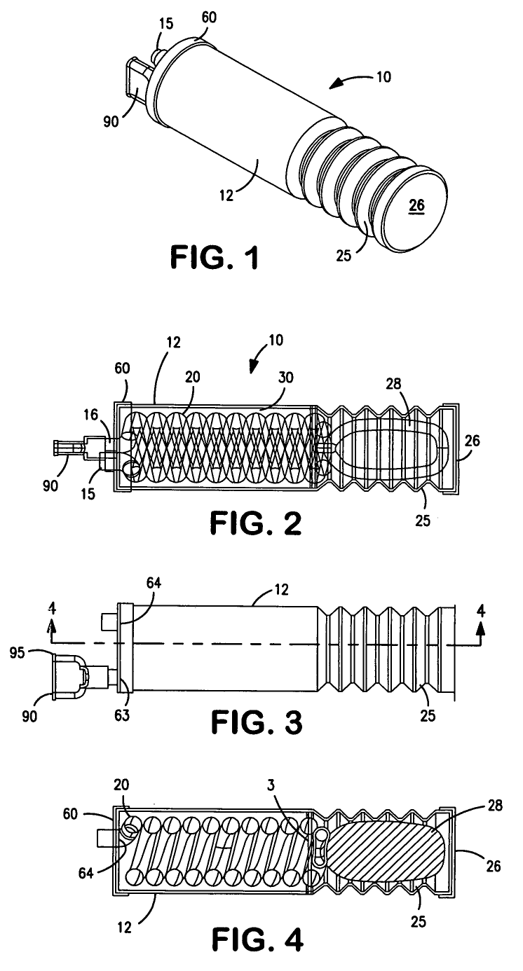

FIG. 1 is a perspective view of a device in accordance with certain embodiments;

FIG. 2 is a cross-sectional view of a device in accordance with certain embodiments;

FIG. 3 is a side view of a device in accordance with certain embodiments;

FIG. 4 is a cross-sectional view taken along line 4-4 of FIG. 3;

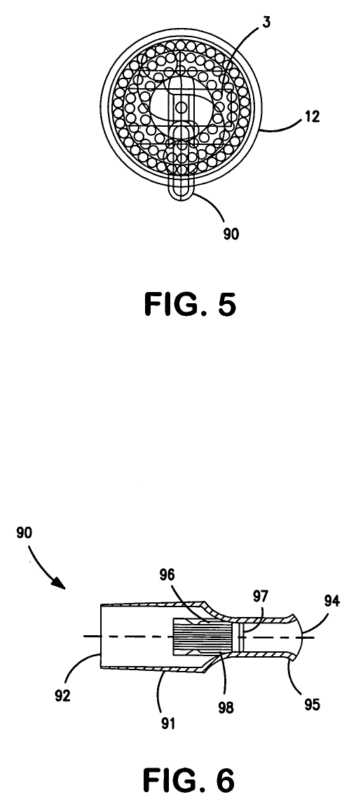

FIG. 5 is a top view of a device in accordance with certain embodiments;

FIG. 6 is a cross-sectional view of a mouthpiece in accordance with certain embodiments;

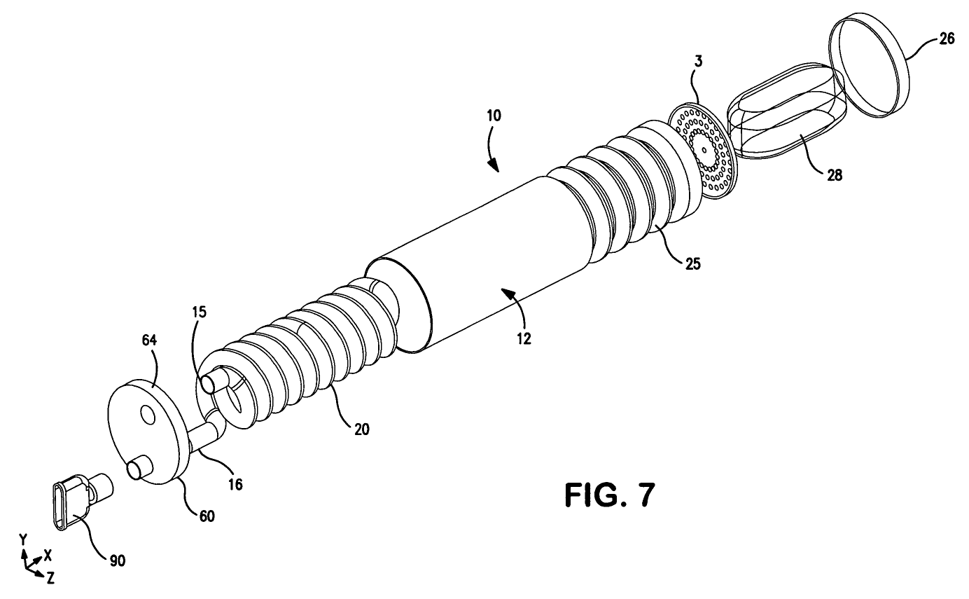

FIG. 7 is an exploded view of a device in accordance with certain embodiments; and

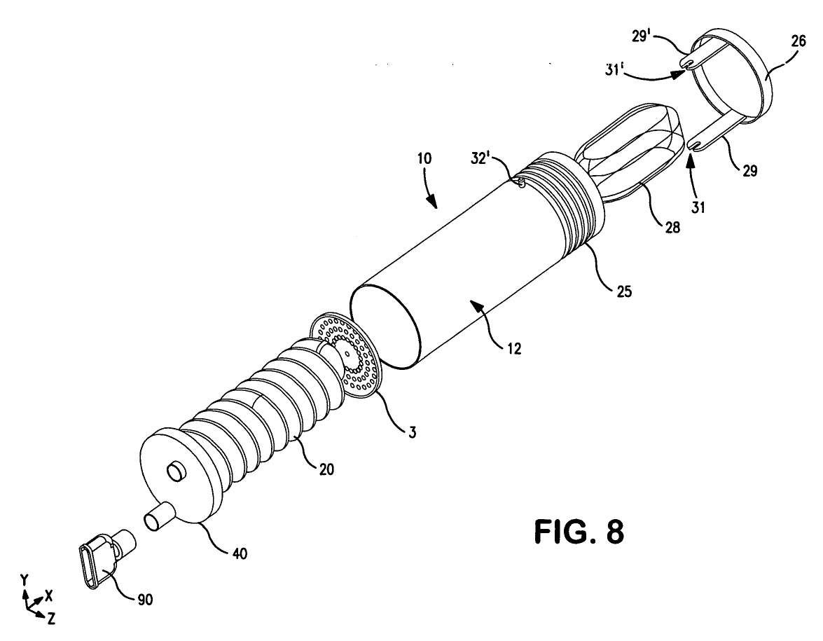

FIG. 8 is an exploded view of a device in accordance with other certain embodiments.

### DETAILED DESCRIPTION OF THE INVENTION

Turning first to FIGS. 1-5 and 7-8, there is shown a device 10 in accordance with certain embodiments of the present invention. The device 10 includes a housing 12 that is shown in the shape of a cylinder. Those skilled in the art will appreciate that other shapes, including rectangles and irregular polygons are suitable and are within the scope of the invention. Preferably the housing 12 is rigid, liquid impervious, and is durable. Suitable materials include plastics, polypropylene, polyvinyl chloride, polytetrafluoroethylene (TEFLON), polyethylene, polycarbonate, Styrofoam, polystyrene, acrylic, metal, glass, etc. The most preferred material is FDA approved low-density polyethylene or high-density polyethylene. The housing 12 can be made to have antimicrobial and/or antibacterial properties, such as by coating or embedding an antimicrobial and/or antibacterial agent therein. The housing 12 defines a volume or chamber that is sized to accommodate, when the device 10 is in its assembled condition, an airflow pathway 20 (best seen in FIG. 2), such as a coil, and a low temperature material or a material adapted to become a low temperature material upon activation.

The airflow pathway 20 is preferably isolated from the air in the remainder of the volume defined by the housing 12, such that air within the pathway 20 does not mix or commingle with air (or liquid) within the remainder of the housing 12. Although a coil is the preferred structure defining the pathway 20, which coil provides a large surface area that is exposed to the low temperature material that is contained in the housing 12, other shapes are within the scope of the present invention. The pathway 20 has a smaller outer diameter than the inner diameter of the housing 12, such that the housing can contain the structure defining the pathway 20, when the device 10 is assembled, and preferably such that a gap 30 between the inner wall of the housing 12 and the structure defining the pathway 20 is maintained (FIG. 2). The pathway 20 preferably has a length similar to the length of the housing 12.

The pathway 20 is defined by a coil that can be made out of a thermally conductive material, such as stainless steel, so that the air inside the coil is quickly and efficiently cooled by the low temperature material contained in the housing 12. The coil shape provides a large surface area for cooling of the air in the pathway 20. Other materials of construction for the coil such as a thermoplastic, or a metal-coated thermoplastic, may be preferred due to their relatively low cost, particularly in view of the disposable nature of the device.

The interior volume or chamber of the housing 12 is configured to hold a low temperature material or coolant (i.e., a material having a lower temperature than the ambient air, preferably at least 10-20° lower, although about 30° lower is acceptable). In the preferred embodiment, the interior volume holds ammonium nitrate that, upon activation, mixes with a fluid to initiate an endothermic reaction, creating a low temperature material. That is, preferably the low temperature material is a material that is normally at room temperature, and that can be caused to lower in temperature by some positive act, such as by mixing with a fluid such as water. Thus, the term “low temperature material” as used herein includes materials that are not initially at a temperature significantly lower than the temperature of the air in the pathway 20 or the ambient, but can be made to have (and preferably retain for at least several minutes) a low temperature relative to the temperature of the air in the pathway 20 or the ambient. Preferably the low temperature material is located in the vicinity of the structure defining the pathway 20, surrounding as much of the surface area of the structure defining the pathway 20 as possible, to enhance the transfer of heat between the structure and the low temperature material. Most preferably, the low temperature material comprises ammonium nitrate crystals. The effective amount of ammonium nitrate crystals (and activating agent) used can be readily determined by those skilled in the art in order to obtain a temperature lowering to about 50-65° F. for about 15-30 minutes.

The structure defining the pathway 20 has a first open end 15 and a spaced second open end 16. The first open end 15 allows for the ingress of air into the passageway 20, such as from the ambient. The second open end 16 allows for the egress of air from the passageway into or in the vicinity of the user's mouth and/or nose. Preferably the first and second open ends are located on the same side of the structure defining the passageway 20, most preferably such that when the structure is contained in the outer housing 12, the first and second open ends are at or near the top of the housing 12 such that they can be in respective fluid communication with an intake port and an outtake port in end cap or cover 60.

Preferably the cover 60 includes an intake port 64 and an outtake port 63. The intake port 64 is in fluid communication with the first open end of the structure defining pathway 20, and the outtake port 63 is in fluid communication with the second open end of the structure defining pathway 20. Accordingly, an airflow loop is defined between the intake port 63, the outtake port 63, and the structure defining the pathway 20. A mouthpiece 90 suitable for use with the device of the invention can be coupled to the outtake port 63. Preferably the mouthpiece 90 is made out of a polyolefin, most preferably polypropylene. One suitable mouthpiece is commercially available from Teleflex Incorporated. Such a mouthpiece 90 has a cylindrical portion 91 having an open end 92. The mouthpiece 90 can be inserted onto the outtake port 63 and fits snuggly thereon by frictional engagement unless it is forceably (such as by manually pulling on the mouthpiece) removed therefrom. The mouthpiece can be removable from the outtake port and can be substituted with a different mouthpiece, depending upon the particular needs of the user.

The distal end of the mouthpiece 90 has an oval shaped opening 94 shaped to the average general contour of a person's mouth (FIG. 6), defined by an outwardly extending flange portion 95 adapted to fit into the patient's mouth and around which the lips of the patient can be positioned so comfortably position and maintain the mouthpiece in the patient's mouth.

Mouthpiece 90 preferably has an internal filter assembly 96, integrally molded therein. In the embodiment shown, the filter assembly 96 includes a front checkerboard grate 97, and opposite side filters 98 tapering outwardly as they extend towards the end 92 of the mouthpiece 90. The filter assembly 96 helps ensure that no particular matter enters the patient's mouth during use of the device, and is shaped to direct the airflow towards the center of the mouthpiece. Those skilled in the art will appreciate that other filter assemblies could be used without departing from the spirit and scope of the invention.

In an alternative embodiment, the mouthpiece 90 can be attached to suitable flex medical tubing (not shown), which tubing is then attached to the device. This allows the device 10 to be positioned further away from the patient's mouth if desired. In yet a further alternative embodiment, a facemask can be placed in fluid communication with the device and can be worn by the user to cover the user's mouth, nose or both. Alternatively still, a nasal cannula can be positioned in fluid communication with the outtake port 63 to direct air into the nose of the patient.

The device 10 includes a flexible member such as a compressible bellows 25 having an internal volume that contains an activating agent, preferably water. Preferably the activating agent is stored in a piercable housing 28, such as a plastic bag. The internal volume of the flexible member is in fluid communication, or can be caused to be in fluid communication, with the internal volume of the housing 12. Compression, twisting, stretching, squeezing or bending of the flexible member, which can be carried out manually, compresses the piercable housing, which breaks the housing and expels the fluid therein. Such manipulation of the bellows also forces the expelled fluid to flow into the housing 12, where it contacts the low temperature material (e.g., ammonium nitrate), causing the material to cool. The bellows 25 is preferably made of a compressible plastic material, such as a low or high density polyolefin, e.g., low or high density polypropylene or low or high density polyethylene, and can include a bottom cap 26 as shown in FIGS. 1 and 4. As best seen in FIG. 4 and 5 and 7 and 8, plastic baffle 3 with a plurality of perforations can be positioned between the housing 12 and the bellows to prevent the ammonium nitrate from displacing from the interior volume of the housing 12 into the interior volume of the bellows. The baffle also serves as a barrier against which the plastic bag in the bellows is compressed upon compression of the bellows. A vent or exhaust port (not shown) or a microfilter 40 FIG. 8) can be located in the device, such as in cover 60, to expel excess air that is forced out of the flexible member 25 upon compression, twisting, stretching or squeezing.

FIG. 8 illustrates an embodiment where the bottom cap 26 is locked onto the device to prevent disassembly or leakage. In the embodiment shown, the bottom cap 26 includes longitudinally extending arms 29, 29′that terminate in respective notched ends 31, 31′. The notched ends 31, 31′ are configured to receive corresponding protrusions 32, 32′ (only one shown) located on the surface of the housing 12 in locking engagement to secure the bottom cap 26 in place.

In use in a first mode of operation, an effective amount of a low temperature material, such as ammonium nitrate, is placed in the interior of the housing 12, and an effective amount of activating agent, such as water, is placed in the interior of the flexible member. The amount of low temperature material is not particularly limited, but should be present in a sufficient amount that upon activation, it cools the airflow passageway (and thus air in that passageway) to a sufficient degree (a cooling of about 10-20 degrees less than ambient) quickly and for a sufficient amount of time, preferably at least about 15-30 minutes. The ammonium nitrate is activated, such as by breaking an inner bag or container within the compressible member (e.g., bellows 25) by flexing the flexible member such as by compression, causing the fluid in the bag to flow into the housing 12 and react with the ammonium nitrate therein. The activation of the low temperature material is preferably carried out just prior to use of the device by the patient. The now cool solution of ammonium nitrate and water flows around the airflow passageway and cools the same. Agitation of the device optionally can be carried out to enhance the cooling. The patient then places his mouth about the mouthpiece 90, and inhales. The inhalation is a driving force that draws ambient air into the device 10 through the intake opening 64, through the pathway 20, where the air in the pathway 20 is cooled by the low temperature material. The now cool air exits the pathway 20 and enters the mouthpiece 90, from which it enters the mouth of the patient, where it cools the throat, larynx, upper respiratory tract, etc. of the patient, providing soothing relief thereto. The intake opening and outtake opening, although in fluid communication via the pathway 20, are segregated; intake air must flow through the pathway 20 (and thus be cooled by the low temperature material) prior to reaching the outtake. The patient may then remove his mouth from the mouthpiece 90 and exhale into the ambient environment. Once the low temperature material is no longer cool relative to the ambient, the apparatus can be properly disposed of.

In an alternative mode of operation, particularly applicable for use with small children (e.g., under 3 years of age), infants or the elderly, an air assist can be provided. In this mode, a driving force, such as a manually operated air bulb, pump or fan, is then placed in fluid communication with the intake opening (such as through suitable tubing (not shown)), and is actuated such that air is forced into the pathway 20, where the air contacts the low temperature material which effectively lowers the temperature of the air. The now cool air exits the pathway 20, flowing through the mouthpiece 90 and into the mouth of the patient. It should be noted that the mouthpiece need not be positioned inside the mouth of the patient; it is sufficient to place the mouthpiece or outtake in proximity to the mouth and/or nose of the patient (e.g., within about 3 inches thereof) to effectively cool the air that the patient is breathing. Indeed, this allows the patient to breath normally without forcing too much cool air into the respiratory system of the patient. It should be noted that the mouthpiece can be omitted entirely in this mode. Alternatively still, a nosepiece such as a nasal cannula (not shown) can be used and can be inserted into the nose of the patient or placed in proximity thereto. The air assist as the driving force for air circulation through the device can be provided alone or in addition to inhalation by the patient.

What is claimed is:

1. Inhaler apparatus, comprising:
a housing defining a volume and adapted to contain a low temperature material;
an airflow pathway positioned in said housing, said airflow pathway defining a passageway isolated from air in the remainder of said housing volume; and
a flexible member defining a volume in fluid communication with said housing and adapted to contain an activating agent for causing said low temperature material to cool.

2. The inhaler apparatus of claim 1, wherein said housing comprises an intake opening in fluid communication with said air flow pathway, and an outtake opening in fluid communication with said air flow pathway.

3. The inhaler apparatus of claim 2, whereby air drawn into said apparatus via said intake opening flows into said air flow pathway, is cooled by said low temperature material, and out said outtake opening.

4. The inhaler apparatus of claim 1, wherein said airflow pathway comprises a coil.

5. The inhaler apparatus of claim 1, further comprising a driving force for forcing air into said intake opening.

6. The inhaler apparatus of claim 1, wherein said low temperature material comprises ammonium nitrate.

7. The inhaler apparatus of claim 6, wherein said activating agent comprises water.

8. The inhaler apparatus of claim 1, wherein said flexible member comprises a bellows.

9. A method of activating a low temperature material to cool air to be inhaled, comprising:
providing a housing having an isolated airflow pathway;
providing a low temperature material in said housing;
providing an activating agent in a flexible member in fluid communication with said housing;
flexing said flexible member to cause said activating agent to flow into said housing and contact said low temperature material to cool said low temperature material, thereby cooling air in said airflow pathway.

10. The method of claim 9, wherein said low temperature material comprises ammonium nitrate.

11. The method of claim 10, wherein said activating agent comprises water.

12. The method of claim 9, wherein said flexing is a compression of said flexible member.

13. The method of claim 9, further comprising inhaling air from said airflow pathway.

14. The method of claim 9, wherein said flexing is a twisting or said flexible member.

15. The method of claim 9, wherein said flexing is a stretching of said flexible member.

16. A method of cooling the airways of an individual, comprising:
providing an inhaler comprising an intake opening and a chamber for a low temperature material,
providing an airflow pathway in said chamber for carrying air isolated from air in the remainder of said chamber;
activating said low temperature material by contacting said low temperature material with an activating agent to cool said low temperature material; and
inhaling ambient air through said intake opening, causing said inhaled air to flow through said airflow pathway where it is cooled by said low temperature material.

17. The method of claim 16, wherein said low temperature material comprises ammonium nitrate.

18. The method of claim 16, wherein said activating agent comprises water.
